(19) United States
(12) Patent Application Publication
(10) Pub. No.: US 2009/0170056 A1
(43) Pub. Date: Jul. 2, 2009

(54) METHOD, MEDIUM, AND APPARATUS FOR PROVIDING EDUCATIONAL MATERIAL IN REMOTE MONITORING SYSTEM

(75) Inventors: Yun-sun NAM, Seongnam-si (KR); Jay-woo Kim, Seongnam-si (KR); Soo-hyun Bae, Seoul (KR); Byung-soo Gim, Yongin-si (KR); Kyu-tae Yoo, Seongnam-si (KR)

Correspondence Address: SUGHRUE MION, PLLC, 2100 PENNSYLVANIA AVENUE, N.W., SUITE 800, WASHINGTON, DC, 20037, US

(73) Assignee: Samsung Electronics Co., Ltd., Suwon-si (KR)

(21) Appl. No.: 12/116,244

(22) Filed: May 7, 2008

(30) Foreign Application Priority Data
Dec. 26, 2007 (KR) 10-2007-0137511

(51) Int. Cl. G09B 19/00; A61B 5/00

(52) U.S. Cl. 434/247; 600/300

(57) ABSTRACT

Provided are a method and an apparatus for providing educational material. The method includes: receiving information about a health state and lifestyle of a patient from a remote terminal at every predetermined period of time; selecting educational material appropriate for the patient from a plurality of pieces of educational material based on the information; and providing the selected educational material to the remote terminal. As a result, educational material appropriate for private characters of a patient can be selected and provided.

### CROSS-REFERENCE TO RELATED PATENT APPLICATION

This application claims the benefit of Korean Patent Application No. 10-2007-0137511, filed on Dec. 26, 2007, in the Korean Intellectual Property Office, the disclosure of which is incorporated herein in its entirety by reference.

### BACKGROUND

1. Field

One or more embodiments of the present invention relate to a method, medium, and apparatus for providing an educational material, and more particularly, to a method, medium, and apparatus for providing educational material for a chronic patient using a remote monitoring system.

2. Description of the Related Art

Researches on remote monitoring systems have been briskly made in the medical technology field. A representative example of such a remote monitoring system includes ubiquitous-Health (u-Health).

Such u-Health refers to a service into which information communication technology and health medical service are combined and provides health medical services of preventions, diagnoses, treatments, and post-managements. Thus, the public, patients, the disabled, or the aged and infirm can periodically check their health states in homes or medical care institutions through wire and wireless communication networks.

For example, u-Health is a system through which chronic disease patients suffering from diabetes, obesity, hypertension, etc. can check their health states using the wireless Internet, personal computers (PCs), mobile phones, etc. anytime anywhere and can be provided with real-time medical examinations from medical institutions.

### SUMMARY

One or more embodiments of the present invention provide a method and an apparatus for providing educational material appropriate for private characters of a chronic disease patient so that the chronic disease patient performs efficient self-management, and a computer-readable medium having a computer readable code to implement the method.

Additional aspects and/or advantages will be set forth in part in the description which follows and, in part, will be apparent from the description, or may be learned by practice of the invention.

According to an aspect of the present invention, there is provided a method of providing educational material including: receiving information about a health state and lifestyle of a patient from a remote terminal at every predetermined period of time; selecting educational material appropriate for the patient from a plurality of pieces of educational material based on the information; and providing the selected educational material to the remote terminal.

According to another aspect of the present invention, there is provided a computer-readable medium having a computer readable code to implement to a method of providing educational material including: receiving information about a health state and lifestyle of a patient from a remote terminal at every predetermined period of time; selecting educational material appropriate for the patient from a plurality of pieces of educational material based on the information; and providing the selected educational material the remote terminal.

According to another aspect of the present invention, there is provided an apparatus of providing educational material including: a patient information storage to store at least one of information about a health state and lifestyle of a patient, genetic information, and medical information; an educational material selector to select educational material appropriate for the patient based on at least one of the information about the health state and the lifestyle of the patient, the genetic information, and the medical information stored in the patient information storage; and an educational material storage to store educational material to be provided and to provide the selected educational material to the patient.

### BRIEF DESCRIPTION OF THE DRAWINGS

These and/or other aspects and advantages will become apparent and more readily appreciated from the following description of the embodiments, taken in conjunction with the accompanying drawings of which:

FIG. 1 is a block diagram of a remote monitoring system according to an embodiment of the present invention;

FIG. 2 is a detailed block diagram of a patient information storage of an educational material provider of FIG. 1;

FIG. 3 is a detailed block diagram of an educational material storage of the educational material provider of FIG. 1;

FIG. 4 is a block diagram illustrating an operation of a complication management unit of the educational material provider of FIG. 1;

FIG. 5 is a flowchart of the operation of the complication management unit of FIG. 4, according to an embodiment of the present invention;

FIG. 6 is a block diagram illustrating an initial operation of a lifestyle management unit of the educational material provider of FIG. 1;

FIG. 7 is a flowchart of the initial operation of the lifestyle management unit of FIG. 6, according to an embodiment of the present invention;

FIG. 8 is a block diagram illustrating a normal operation of the lifestyle management unit of the educational material provider of FIG. 1;

FIGS. 9A and 9B are flowcharts of the normal operation of the lifestyle management of FIG. 8, according to an embodiment of the present invention; and

FIG. 10 is a flowchart of a method of providing educational material according to an embodiment of the present invention.

### DETAILED DESCRIPTION OF THE EMBODIMENTS

Reference will now be made in detail to embodiments, examples of which are illustrated in the accompanying drawings, wherein like reference numerals refer to the like elements throughout. In this regard, embodiments of the present invention may be embodied in many different forms and should not be construed as being limited to embodiments set forth herein. Accordingly, embodiments are merely described below, by referring to the figures, to explain aspects of the present invention.

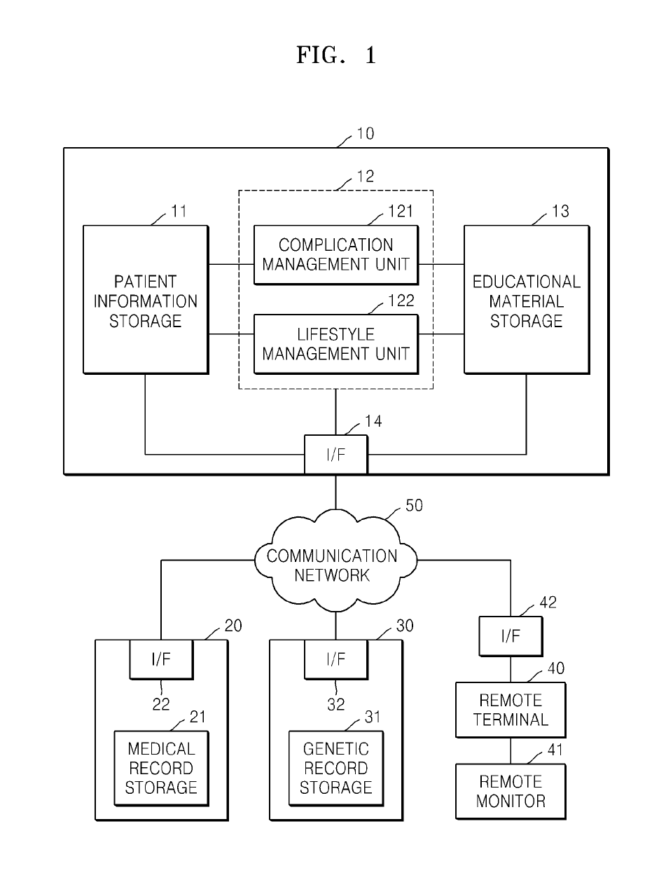

FIG. 1 is a block diagram of a remote monitoring system according to an embodiment of the present invention.

Referring to FIG. 1, the remote monitoring system may include an educational material provider 10, a medical record provider 20, a genetic test provider 30, and a remote terminal 40. The educational material provider 10, the medical record provider 20, the genetic test provider 30, and the remote terminal 40 may be connected to a communication network 50. Here, the communication network 50 may be a path through which a plurality of individuals communicate information to one another, i.e., a wire and wireless network such as the Internet, a telephone line, or the like.

The educational material provider 10 may include a patient information storage 11, an educational material selector 12, and an educational material storage 13 and be connected to the communication network 50 through a communication interface 14.

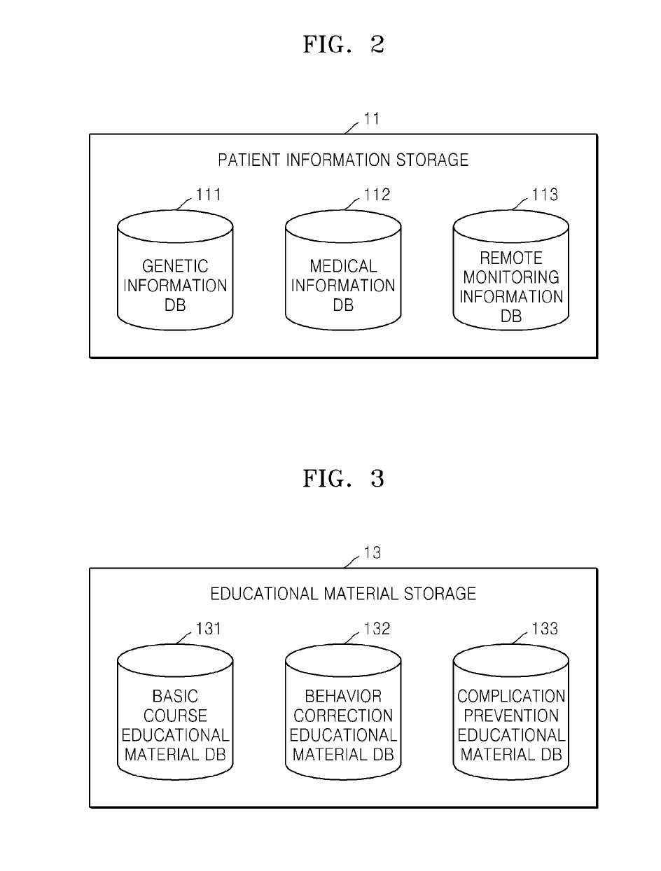

The patient information storage 11 may store patient-related information that is provided from the medical record provider 20, the genetic test provider 30, and the remote terminal 40 through the communication network 50. The patient information storage 11 will be described in detail later with reference to FIG. 2.

The educational material selector 12 may select a type, a transmission frequency, etc. of educational material appropriate for a patient based on the patient-related information stored in the patient information storage 11. In more detail, the educational material selector 12 may include a complication management unit 121 and a lifestyle management unit 122.

The complication management unit 121 may calculate a complication risk of a chronic disease of the patient based on genetic information, a health state, a family history, etc. of the patient stored in the patient information storage 11. The complication management unit 121 may also select a type of complication prevention educational material appropriate for the patient according to the calculation result and control a transmission frequency of the complication prevention educational material.

The lifestyle management unit 122 may analyze lifestyle of the patient based on remote-monitored results stored in the patient information storage 11, select behavior correction educational material about the lifestyle according to the analysis result, and control a transmission frequency of the behavior correction educational material.

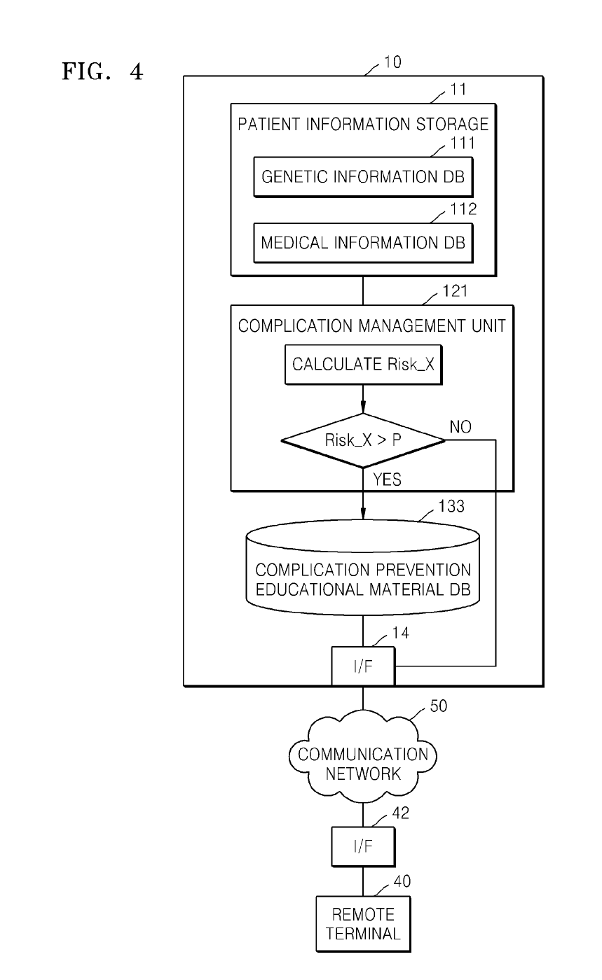

The educational material storage 13 may store educational material that is to be provided to the patient by remote control and provide the educational material to the patient through the communication network 50 according to the selection result of the educational material selector 12. The educational material storage 13 will be described in detail later with reference to FIG. 3.

The medical record provider 20 may store a medical examination result of the health state of the patient and provide the medial examination result to the educational material provider 10 through the communication network 50 when necessary. In more detail, the medical record provider 20 may include a medical record storage 21 and a communication interface 22. The medical record storage 21 may store the medical examination result of the health state of the patient, and the communication interface 22 may interface the medical record provider 20 with the communication network 50.

Here, the medical examination result stored in the medical record storage 21 may include a physical character, a family history, lifestyle, medical data, etc. Such information stored in the medical record storage 21 is not frequently changed and thus is not required to be continuously provided from the patient.

The genetic test provider 30 may store a genetic test result of the patient and provide the genetic test result to the educational material provider 10 through the communication network 50 when necessary. In more detail, the genetic test provider 30 may include a genetic record storage 31 and a communication interface 32. The genetic record storage 31 may store the genetic test result of the patient, and the communication interface 32 may interface the genetic test provider 30 with the communication network 50. Information stored in the genetic record storage 31 is not changed and thus is not required to be continuously provided from the patient.

The remote terminal 40 may periodically provide results monitored by a remote monitor 41 to the educational material provider 10 through the communication network 50. For example, the remote terminal 40 may provide the monitored results to the educational material provider 10 everyday. Here, the remote monitor 41 may measure and monitor the health state of the patient such as a blood sugar level of the patient, the lifestyle of the patient including meal menu and quantity, exercise type and time, an attitude, etc. of the patient, and so on through questions and answers, etc.

The remote terminal 40 may be periodically provided with educational material necessary for the patient from the educational material provider 10 through the communication network 50. For example, the remote terminal 40 may be provided with the educational material necessary for the patient everyday. As a result, the patient can be periodically provided with remote medical service without visiting a medical institution.

FIG. 2 is a detailed block diagram of the patient information storage 11 of the educational material provider 10 of FIG. 1.

Referring to FIGS. 1 and 2, the patient information storage 11 may include a genetic information database (DB) 111, medical information DB 112, and remote monitoring information DB 113.

The genetic information DB 111 may store information provided from the genetic test provider 30 of FIG. 1. In more detail, the genetic information DB 111 may store information indicating whether the patient has genetic variants of complications of a chronic disease of the patient such as diabetes, a heart disease, hypertension, etc.

The medical information DB 112 may store information provided from the medical record provider 20 of FIG. 1. In more detail, the medical information DB 112 may store the physical character, the family history, and the lifestyle of the patient, medical information, etc.

Here, the physical character of the patient may include an age, the gender, a human race, a height, a weight, etc. of the patient. Also, the family history may include medical histories of the family or intimate relations of the patient or people living with the patient, i.e., their health states, suffering diseases, hereditary diseases, death causes, etc. Thus, the family history is used to cure the patient. In detail, the family history may include information indicating whether parents or brothers and sisters of the patient suffer from chronic diseases such as diabetes, heart diseases, hypertensions, etc. Also, the lifestyle may include basic lifestyle such as a smoking or drinking habit of the patient, etc. The medical information may include a test result of glycosylated hemoglobin (HbA1C) that is an index indicating a rate of sugar attached to hemoglobin of the patient, a blood pressure, a result of a lipid test for measuring a blood lipid level, etc.

The remote monitoring DB 113 may store information provided from the remote terminal 40 of FIG. 1. In more detail, the remote monitoring information DB 113 may be provided with and store information about the health state such as the blood sugar level of the patient, information about the meal menu and quantity of the patient, the exercise type and time, and other lifestyles, etc. at every predetermined period of time by remote control. Here, the remote monitoring information DB 113 may store the information that is provided from the remote terminal 40 at the every predetermined period of time and thus can be updated at every predetermined period of time. For example, the every predetermined period of time may be everyday or every week.

FIG. 3 is a detailed block diagram of the educational material storage 13 of the educational material provider 10 of FIG. 1.

Referring to FIGS. 1 and 3, the educational material storage 13 may include a basic course educational material DB 131, a behavior correction educational material DB 132, and a complication prevention educational material DB 133.

The basic course educational material DB 131 may store educational material that is commonly transmitted to all of patients who use the remote monitoring system. Here, the commonly transmitted educational material may include symptoms and management methods of chronic diseases, etc. For example, if the remote monitoring system is used for diabetes, the commonly transmitted educational material may include meanings, symptoms, complications, emergency coping methods, management methods (e.g., dietetic therapy, exercise methods, self-blood sugar measurement methods, periodical inspections, feet management methods, etc) of diabetes, etc.

The behavior correction educational material DB 131 may store material about behaviors to be corrected based on the remote-monitored information stored in the patient information storage 11. Here, the material may be to be provided to individual patients. For example, the behavior correction educational material DB 131 stores correction educational material about meal menus and quantity, exercise types and time, fighting attitudes against diseases, self-blood sugar measurement methods, medicine taking methods, insulin administering methods, etc.

The complication prevention educational material DB 133 may store material about complications of chronic diseases based on the genetic information and the medical information stored in the patient information storage 11. Here, the material may be to be provided to individual patients. For example, if the remote monitoring system is used for diabetes, the complication prevention educational material DB 133 stores material about hypertension, high cholesterol, cardiovascular diseases, retinopathy, cardiac disorders, nerve complications of motive, sensory, and autonomic nerves that are complications of the diabetes, etc.

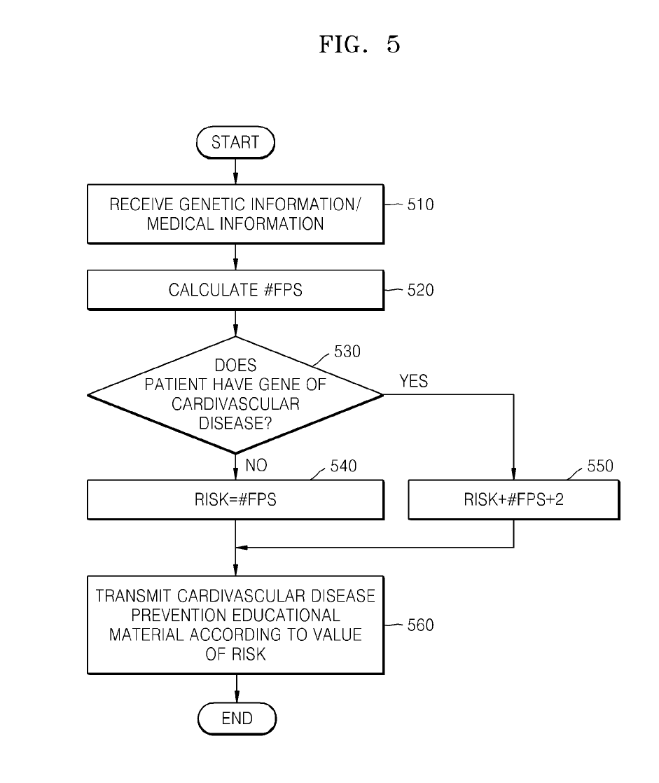

FIG. 4 is a block diagram illustrating an operation of the complication management unit 121 of the educational material provider 10 of FIG. 1.

Referring to FIG. 4, the educational material provider 10 may include the patient information storage 11 having the genetic information DB 111 and the medical information DB 112, and the complication prevention management unit 121, and the complication prevention educational material DB 133. The educational material provider 10 may also transmit educational material to the remote terminal 40 through the communication network 50. The operation of the complication management unit 121 will now be described.

The complication management unit 121 may calculate a risk Risk_X to a complication of a specific chronic disease X of a patient according to information stored in the genetic information DB 111 and the medical information DB 112. Here, the information stored in the genetic information DB 111 and the medical information DB 112 may include genetic information, health states, family histories, etc. of the patient. The specific chronic disease X may be a chronic disease such as diabetes, a heart disease, hypertension, or the like.

The complication management unit 121 may determines whether the risk Risk_X is greater than a predetermined value P.

If the risk Risk_X is not greater than the predetermined value P, the patient is at a low risk to the complication. In this case, the complication management unit 121 may inform the remote terminal 40 through the communication network 50 that the patient is at the low risk to the complication of the specific chronic disease X.

If the risk Risk_X is greater than the predetermined value P, the patient is at a high risk to the complication. In this case, the complication management unit 121 selects corresponding complication prevention educational material and controls a transmission frequency of the corresponding complication prevention educational material.

The complication prevention educational material DB 133 may transmit the corresponding complication prevention educational material to the remote terminal 40 through the communication network 50 according to the complication prevention educational material and the transmission frequency selected by the complication management unit 121. As a result, the patient can be provided with complication prevention material appropriate for private characters of the patient by remote control.

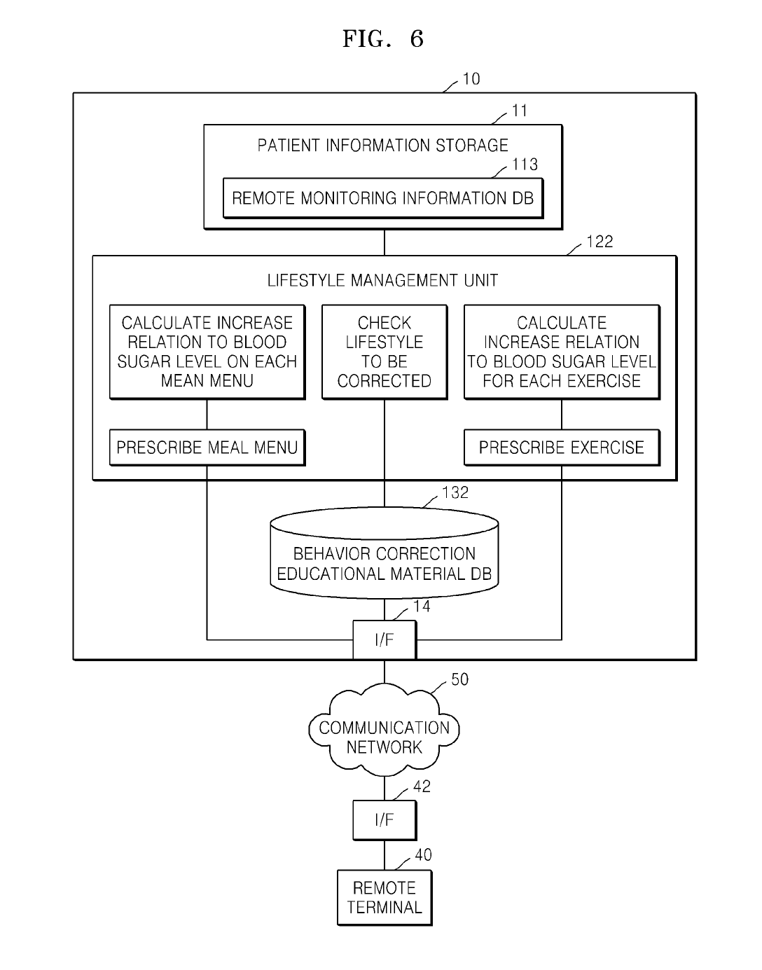

FIG. 5 is a flowchart of the operation of the complication management unit 121 of FIG. 4, according to an embodiment of the present invention.

Here, there will be described the operation of the complication management unit 121 that is performed if a complication is a cardiovascular disease. However, it will be understood by those of ordinary skill in the art that the cardiovascular disease is only an example of a complication.

Referring to FIG. 5, in operation 510, the complication management unit 121 receives the genetic information and the medical information from the patient information storage 11.

In operation 520, the complication management unit 121 calculates a Framingham Point Score (#FPS). Here, Framingham Point Score (#FPS) is used to diagnose a risk to a cardiovascular disease, i.e., is a method of calculating a risk to a cardiovascular disease according to age, total cholesterol (TCHO), smoking, high-density lipoprotein (HDL), and a blood pressure.

In operation 530, the complication management unit 121 determines whether a patient has a genetic variant of a cardiovascular disease.

If the patient does not have the genetic variant of the cardiovascular disease, the complication management unit 121 sets the risk to the cardiovascular disease to the Framingham Point Score (#FPS) in operation 540.

If the patient has the genetic variant of the cardiovascular diseases, in operation 550, the complication management unit 121 sets the risk to the cardiovascular disease to a value that is greater than the Framingham Point Score (#FPS) by a predetermined value. For example, the predetermined value may be “2.” In this case, the risk to the cardiovascular disease may be a value that is 2 greater than the Framingham Point Score (#FPS).

In operation 560, the complication management unit 121 transmits cardiovascular disease prevention educational material to the patient according to the value of the risk to the cardiovascular disease set in operation 540 or 550 at every predetermined period of time. For example, the complication management unit 121 may monthly transmit the cardiovascular disease prevention educational material according to the value of the risk to the cardiovascular disease set in operation 540 or 550.

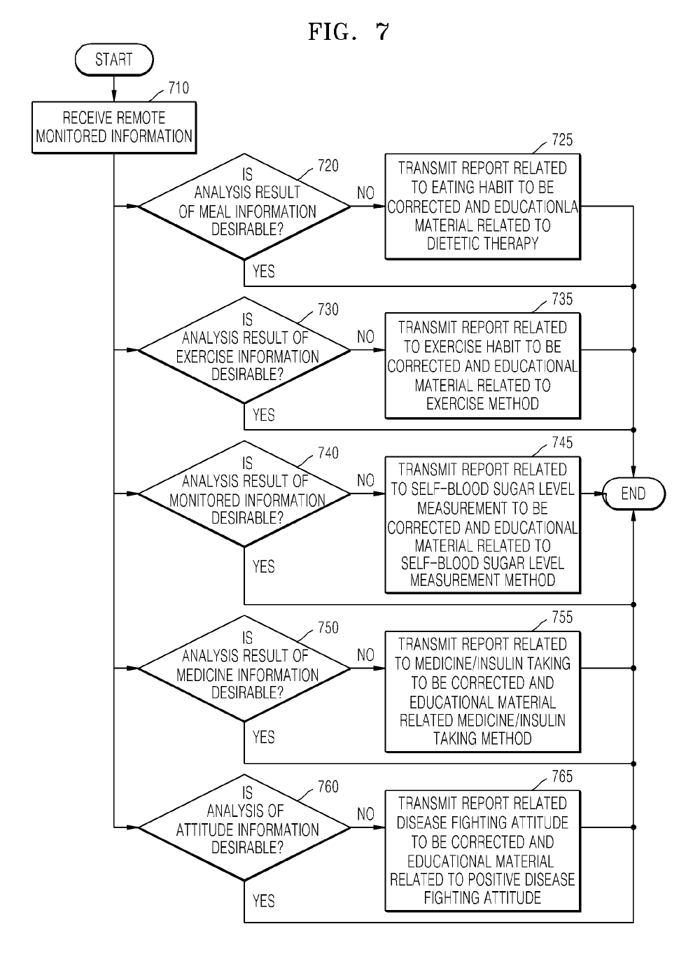

FIG. 6 is a block diagram illustrating an initial operation of the lifestyle management unit 122 of the educational material provider 10 of FIG. 1.

Referring to FIG. 6, the educational material provider 10 may include the patient information storage 11 having the remote monitoring information DB 113, the lifestyle management unit 122, and the behavior correction educational material DB 132. The educational material provider 10 may also transmit educational material to the remote terminal 40 through the communication network 50. The initial operation of the lifestyle management unit 122 will now be described.

The lifestyle management unit 122 may wholly analyze lifestyle of a patient for an initial period of time, wherein the lifestyle is registered in the remote monitoring system by the patient. In detail, the lifestyle management unit 122 may calculate an increase relation to a blood sugar level on each meal menu and an increase relation to a blood sugar level for each exercise and checks lifestyle to be corrected, according to information stored in the remote monitoring information DB 113.

The lifestyle management unit 122 may prescribe appropriate meal menus for the patient based on the increase relation to the blood sugar level on each meal menu and appropriate exercises for the patient based on the increase relation to the blood sugar level for each exercise. The lifestyle management unit 122 may select corresponding behavior correction educational material based on the lifestyle to be corrected and control a transmission frequency of the corresponding behavior correction educational material.

The behavior correction educational material DB 132 may transmit behavior correction educational material to the remote terminal 40 through the communication network 50 according to the behavior correction educational material and transmission frequency selected by the lifestyle management unit 122. As a result, the patient can be provided with behavior correction educational material appropriate for private characters of the patient by remote control.

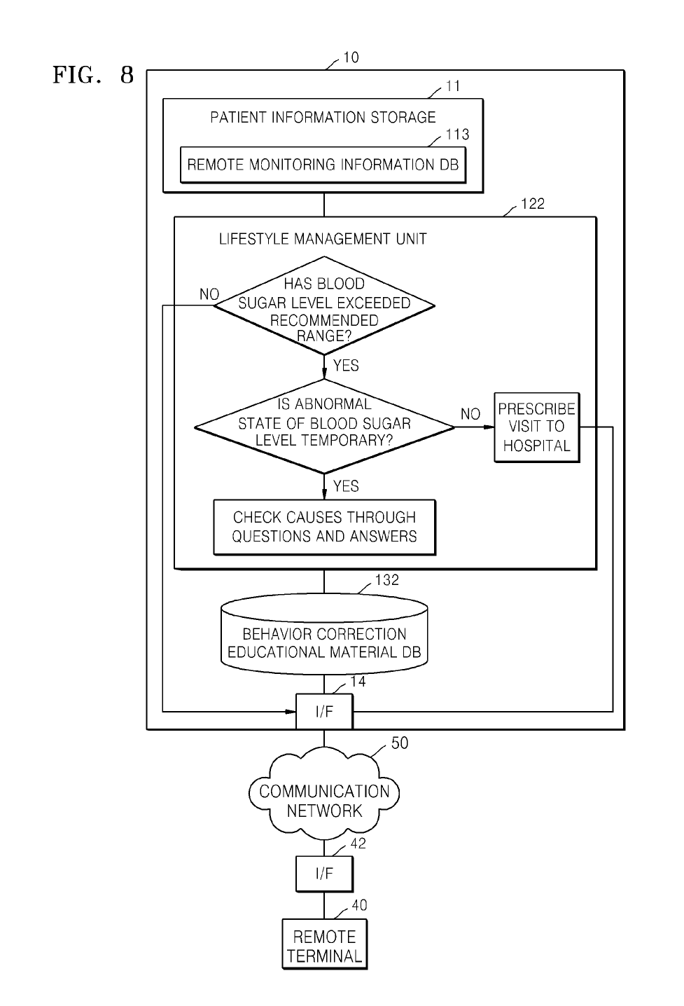

FIG. 7 is a flowchart of the initial operation of the lifestyle management unit 122 of FIG. 6, according to an embodiment of the present invention.

Referring to FIG. 7, in operation 710, the lifestyle management unit 122 receives remote-monitored information from the patient information storage 11.

In operation 720, the lifestyle management unit 122 analyzes information about meal menu and quantity of the patient and determines whether the analysis result is desirable. In more detail, the lifestyle management unit 122 uses information about the meal menu and quantity of the patient to determine answers to items of “Does the patient eat at fixed times?,” “Does the patient eat slowly?,” “Does the patient take a recommended range of carbohydrates?,” “Does the patient take a recommended quantity of fat?,” “Does the patient take a bit of salt?,” “Does the patient take a sufficient quantity of cellulose?,” etc. If the answer to at least of the items is “No,” the lifestyle management unit 122 goes to operation 725. If the answers to all of the items are “Yes,” the present process ends.

In operation 725, the lifestyle management unit 122 and the educational material storage 13 respectively transmit report related to dietetic therapy to be corrected and dietetic therapy management educational material to the patient.

In operation 730, the lifestyle management unit 122 analyzes information about exercise type and time of the patient and determines whether the analysis result is desirable. In more detail, the lifestyle management unit 122 uses the information about the exercise type and time of the patient to determine answers to items of “Does the patient consumes a recommended range of calorie?,” “Doest the patient exercise at fixed time?,” and “Is the number of heartbeats is within a maximum number between 50% and 70% during exercise?,” etc. If the answer to at least one of the items is “No,” the lifestyle management unit 122 goes to operation 735. If the answers to all of the items are “Yes,” the present process ends.

In operation 735, the lifestyle management unit 122 and the educational material storage 13 respectively transmit report related to exercise habits to be corrected and educational material related to exercise methods to the patient.

In operation 740, the lifestyle management unit 122 analyzes monitored information and determines whether the analysis result is desirable. For example, if the remote monitoring system is used for diabetes, the remote monitor 41 can take blood from the patient to measure a blood sugar level. Here, there is an analysis of whether the measurement of the blood sugar level is being properly performed. In more detail, the lifestyle management unit 122 uses the monitored information to determine answers to items of “Is the blood sugar level is taken at fixed time?,” “Does the patient check a validity term of a strip of the blood sugar level?,” “Does the patient keep a measurer clear?,” “Does the patient take a sufficient quantity of blood?,” etc. If the answer to at least one of the items is “No,” the lifestyle management unit 122 goes to operation 745. If the answers to all of the items are “Yes,” the present process ends.

In operation 745, the lifestyle management unit 122 and the educational material storage 13 respectively transmit report related to self-blood measurements to be corrected and educational material related to self-blood sugar measurement to the patient.

In operation 750, the lifestyle management unit 122 analyzes information about medicine taking of the patient and determines whether the analysis result is desirable. In more detail, the lifestyle management unit 122 uses the information about medicine taking of the patient to determine answers to items of “Does the patient take medicine at fixed time?,” “Does the patient take a fixed quantity of medicine?,” “Is the medicine of the patient free of side effects?,” etc. If the answer to at least one of the items is “No,” the lifestyle management unit 122 goes to operation 755. If the answers to all of the items are “Yes,” the present process ends.

In operation 755, the lifestyle management unit 122 and the educational material storage 13 respectively transmit report related to medicine or insulin taking to be corrected and educational material related to medicine or insulin taking methods to the patient.

In operation 760, the lifestyle management unit 122 analyzes information about attitudes of the patient and determines whether the analysis result is desirable. In more detail, the lifestyle management unit 122 uses the information about the attitudes of the patient to determine answers to items of “Is the patient generally in a good mood?,” “Does that patient think that the patient is able to overcome diabetes?,” “Does the patient overcome stress within a short time?,” etc. If the answer to at least one of the items is “No,” the lifestyle management unit 122 goes to operation 765. If the answers to all of the items are “Yes,” the present process ends.

In operation 765, the lifestyle management unit 122 and the educational material storage 13 respectively transmit report related to disease fighting attitudes to be corrected and educational material related to positive disease fighting attitudes to the patient.

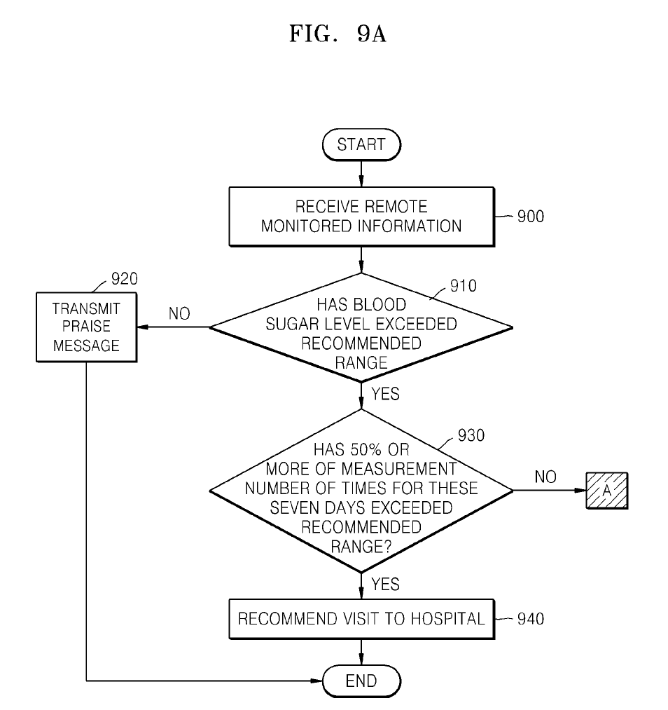

FIG. 8 is a block diagram illustrating a normal operation of the lifestyle management unit 122 of the educational material provider 10 of FIG. 1.

Referring to FIG. 8, the educational material provider 10 may include the patient information storage 11 having the remote monitoring information DB 113, the lifestyle management unit 122, and the behavior correction educational material DB 132. The educational material provider 10 may also transmit educational material to the remote terminal 40 through the communication network 50. The normal operation of the lifestyle management unit 12 will now be described.

After a patient registers information about a blood sugar level of the patient in the remote monitoring system and then a predetermined period of time elapses, the lifestyle management unit 122 determines whether the blood sugar level has exceeded a recommended range, with reference to information monitored by the remote monitoring system.

If the blood sugar level of the patient is within the recommended range, the lifestyle management unit 122 may transmit information about a normal state of the blood sugar level of the patient to the remote terminal 40 through the communication network 50. If the blood sugar level of the patient has exceeded the recommended range, the lifestyle management unit 122 determines whether the blood sugar level of the patient has temporarily exceeded the recommended range.

If an abnormal state of the blood sugar level of the patient is not temporary, the lifestyle management unit 122 may prescribe a visit to a hospital for the patient and transmit information about the visit to the remote terminal 40 through the communication network 50. If the abnormal state of the blood sugar level of the patient is temporary, the lifestyle management unit 122 checks causes of the abnormal state of the blood sugar level through questions and answers.

The lifestyle management unit 122 selects behavior correction educational material for the patient based on the checked causes and controls a transmission frequency of the behavior correction educational material.

The behavior correction educational material DB 132 transmits the behavior correction educational material to the remote terminal 40 through the communication network 50 according to the behavior correction educational material and transmission frequency selected by the lifestyle management unit 122. As a result, the patient can be provided with behavior correction educational material appropriate for private characters of the patient by remote control.

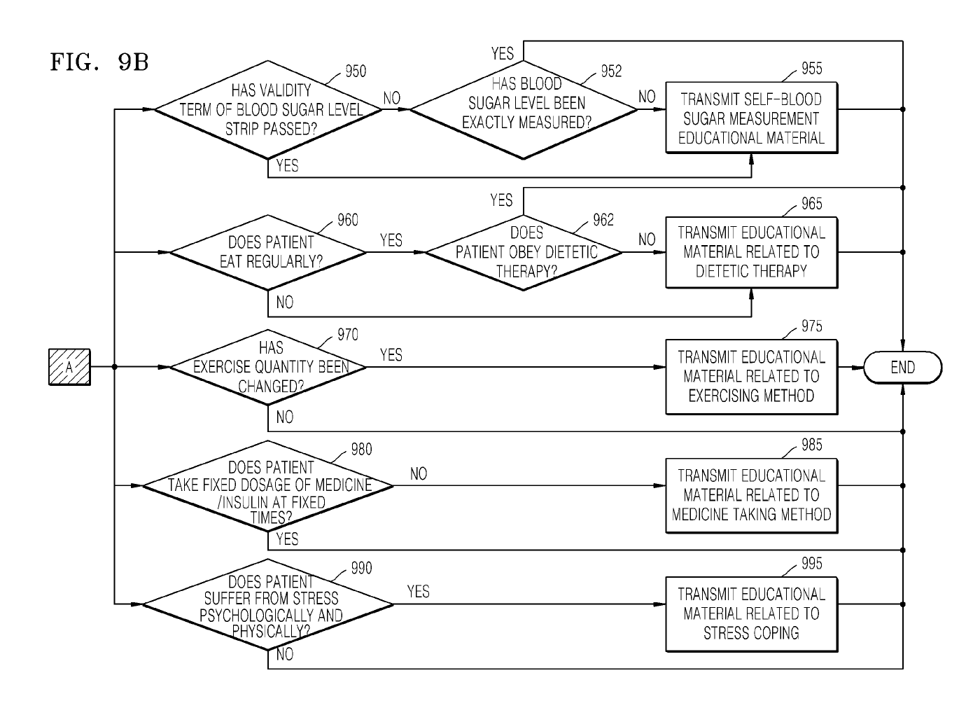

FIGS. 9A and 9B are flowcharts of the normal operation of the lifestyle management unit 122 of FIG. 8, according to an embodiment of the present invention.

Referring to FIGS. 9A and 9B, in operation 900, the lifestyle management unit 122 receives remote-monitored information from the patient information storage 11.

In operation 910, the lifestyle management unit 122 analyzes information about a blood sugar level of a patient and determines whether the blood sugar level has exceeded a recommended range. If the blood sugar level has not exceeded the recommended range, the lifestyle management unit 122 goes to operation 920. If the blood sugar level has exceeded the recommended range, the lifestyle management unit 122 goes to operation 930.

If the blood sugar level of the patient has not exceeded the recommended range, the lifestyle management unit 122 transmits a praise message to the patient through the communication network 50 in operation 920.

In operation 930, the lifestyle management unit 122 determines whether predetermined number of times or more of the number of times blood sugar level is measured for a predetermined period of time have exceeded a recommended range. In more detail, the lifestyle management unit 122 determines whether 50% or more of the number of times blood sugar level is measured for these seven days has exceeded the recommended range. If 50% or more of the number of times has not exceeded the recommended range, the lifestyle management unit 122 goes to “A.” If 50% or more of the number of times has exceeded the recommended range, the lifestyle management unit 122 goes to operation 940.

If 50% or more of the number of times has exceeded the recommended range, the lifestyle management unit 122 transmits an advice message for a visit to a hospital to the patient through the communication network 50 in operation 940.

In operation 950, the lifestyle management unit 122 determines whether a validity term of a blood sugar level strip has been expired. If the validity term of the blood sugar level strip has not been expired, the lifestyle management unit 122 goes to operation 952. If the validity term of the blood sugar level strip has been expired, the lifestyle management unit 122 goes to operation 955.

If the validity term of the blood sugar level strip has not been expired, the lifestyle management unit 122 determines whether the blood sugar level has been exactly measured, in operation 952. If the blood sugar level has not been exactly measured, the lifestyle management unit 122 goes to operation 955. If the blood sugar level has been exactly measured, the present process ends.

In operation 955, the lifestyle management unit 122 selects self-blood sugar measurement educational material from the behavior correction educational material DB 132 and transmits the self-blood sugar measurement educational material to the patient.

In operation 960, the lifestyle management unit 122 determines whether the patient has regularly eaten meals. If the patient has regularly eaten the meals, the lifestyle management unit 122 goes to operation 962. If the patient has not regularly eaten the meals, the lifestyle management unit 122 goes to operation 965.

In operation 962, the lifestyle management unit 122 determines whether the patient has obeyed a dietetic therapy. If the patient has not obeyed the dietetic therapy, the lifestyle management unit 122 goes to operation 965. If the patient has obeyed the dietetic therapy, the present process ends.

In operation 965, the lifestyle management unit 122 selects dietetic therapy-related educational material from the behavior correction educational material DB 132 and transmits the dietetic therapy-related educational material to the patient.

In operation 970, the lifestyle management unit 122 determines whether a quantity of exercise of the patient has been changed. If the quantity of exercise of the patient has been changed, the lifestyle management unit 122 goes to operation 975. If the quantity of exercise of the patient has not been changed, the present process ends.

In operation 975, the lifestyle management unit 122 selects an exercising method-related educational material from the behavior correction educational material DB 132 and transmits the exercising method-related educational material to the patient.

In operation 980, the lifestyle management unit 122 determines whether the patient has taken a dosage of medicine or insulin at fixed times. If the patient has not taken the dosage of medicine or insulin at the fixed times, the lifestyle management unit 122 goes to operation 985. If the patient has taken the dosage of medicine or insulin at the fixed times, the present process ends.

In operation 985, the lifestyle management unit 122 selects medicine taking method-related educational material from the behavior correction educational material DB 132 and transmits the medicine taking method-related educational material to the patient.

In operation 990, the lifestyle management unit 122 determines whether the patient suffers from stress psychologically and physically. If the patient suffers from stress psychologically and physically, the lifestyle management unit 122 goes to operation 995. If the patient does not suffer from stress psychologically and physically, the present process ends.

In operation 995, the lifestyle management unit 122 selects stress coping method-related educational material from the behavior correction educational material DB 132 and transmits the stress coping method-related educational material to the patient.

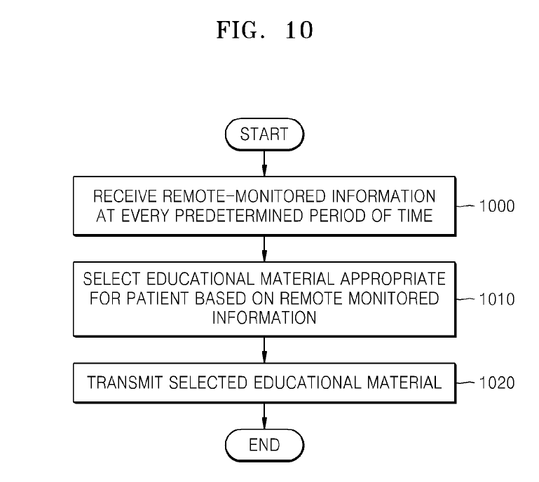

FIG. 10 is a flowchart of a method of providing educational material according to an embodiment of the present invention.

Referring to FIG. 10, the method includes operations that are processed by the educational material provider 10 illustrated in FIGS. 1 through 3 in time series. Therefore, a few of contents will be omitted. However, if the omitted contents correspond to ones of the above-described contents of the educational material provider 10 of FIGS. 1 through 3, they will be applied to the method of the present embodiment.

In operation 1000, the remote monitoring information DB 113 of the patient information storage 10 updates remote-monitored information about health states and lifestyle of a patient at every predetermined period of time.

In operation 1010, the educational material selector 12 selects educational material appropriate for the patient based on at least one of genetic information, medical information, and the updated remote-monitored information about the patient.

In operation 1020, the educational material storage 13 provides the patient with the educational material selected by the educational material selector 12.

In addition to the above described embodiments, embodiments of the present invention can also be implemented through computer readable code/instructions in/on a medium, e.g., a computer readable medium, to control at least one processing element to implement any above described embodiment. The medium can correspond to any medium/media permitting the storing and/or transmission of the computer readable code.

The computer readable code can be recorded/transferred on a medium in a variety of ways, with examples of the medium including recording media, such as magnetic storage media (e.g., ROM, floppy disks, hard disks, etc.) and optical recording media (e.g., CD-ROMs, or DVDS), and transmission media such as carrier waves, as well as through the Internet, for example. Thus, the medium may further be a signal, such as a resultant signal or bitstream, according to embodiments of the present invention. The media may also be a distributed network, so that the computer readable code is stored/transferred and executed in a distributed fashion. Still further, as only an example, the processing element could include a processor or a computer processor, and processing elements may be distributed and/or included in a single device.

As described above, in a method and an apparatus for providing educational material in a remote monitoring system according to one or more embodiments of the present invention, remote-monitored information about health states and lifestyle of a patient can be updated at every predetermined period of time. Educational material appropriate for the patient can be selected based on at least one of genetic information, medical information, and the updated remote-monitored information about the patient. The educational material can be provided to the patient. Thus, educational material appropriate for private characters of the patient can be selected and provided. As a result, an efficient self-management of a chronic disease patient whose lifestyle should be corrected and self-managed can be induced.

While aspects of the present invention has been particularly shown and described with reference to differing embodiments thereof, it should be understood that these exemplary embodiments should be considered in a descriptive sense only and not for purposes of limitation. Any narrowing or broadening of functionality or capability of an aspect in one embodiment should not considered as a respective broadening or narrowing of similar features in a different embodiment, i.e., descriptions of features or aspects within each embodiment should typically be considered as available for other similar features or aspects in the remaining embodiments.

Thus, although a few embodiments have been shown and described, it would be appreciated by those skilled in the art that changes may be made in these embodiments without departing from the principles and spirit of the invention, the scope of which is defined in the claims and their equivalents.

What is claimed is:

1. A method of providing educational material, the method comprising:
receiving information about a health state and lifestyle of a patient from a remote terminal at every predetermined period of time;
selecting educational material appropriate for the patient from a plurality of pieces of educational material based on the information; and
providing the selected educational material to the remote terminal.

2. The method of claim 1, wherein the selection of the educational material comprises:
analyzing the lifestyle of the patient based on the information; and
selecting behavior correction educational material about the lifestyle according to the analysis result.

3. The method of claim 2, wherein the selection of the educational material further comprises controlling a transmission frequency of the behavior correction educational material according to the analysis result.

4. The method of claim 2, wherein the information about the health state of the patient comprises a blood sugar level of the patient, and the information about the lifestyle of the patient comprises at least one of meal menu and quantity, exercise type and time, and an attitude of the patient,
wherein the analysis of the lifestyle of the patient comprises calculating at least one of a relationship between the meal menu and quantity and the blood sugar level of the patient and a relationship between the exercise type and time and the blood sugar level of the patient.

5. The method of claim 4, further comprising prescribing meal menu and exercise appropriate for the patient according to the calculation result.

6. The method of claim 2, wherein the information about the health state of the patient comprises a blood sugar level of the patient,
wherein the analysis of the lifestyle of the patient comprises: analyzing causes through questions and answers by remote control when the blood sugar level of the patient exceeds a predetermined range.

7. The method of claim 6, further comprising: transmitting a praise message to the patient when the blood sugar level does not exceed the predetermined range.

8. The method of claim 7, further comprising: transmitting an advice message for a visit to a hospital to the patient when the blood sugar level of the patient exceeds the predetermined range a pre-set number of times or more for a pre-set period of time.

9. The method of claim 1, further comprising receiving at least one of genetic information and medical information about the patient.

10. The method of claim 9, wherein the selection of the educational material comprises:
calculating a complication risk of the patient based on at least one of the genetic information and the medical information; and
selecting complication prevention educational material when the complication risk is greater than or equal to a predetermined value.

11. The method of claim 10, wherein the selection of the educational material further comprises controlling a transmission frequency of the complication prevention educational material according to the complication risk.

12. A computer-readable medium having a computer readable code to implement a method of providing educational material comprising:
receiving information about a health state and lifestyle of a patient from a remote terminal at every predetermined period of time;
selecting educational material appropriate for the patient from a plurality of pieces of educational material based on the information; and
providing the selected educational material to the remote terminal.

13. An apparatus of providing educational material, the apparatus comprising:
a patient information storage to store at least one of information about a health state and lifestyle of a patient, genetic information, and medical information;
an educational material selector to select educational material appropriate for the patient based on at least one of the information about the health state and the lifestyle of the patient, the genetic information, and the medical information stored in the patient information storage; and
an educational material storage to store educational material to be provided and to provide the selected educational material to the patient.

14. The apparatus of claim 13, wherein the information about the health state and the lifestyle of the patient stored in the patient information storage is updated at every predetermined period of time.

15. The apparatus of claim 13, wherein the educational material storage comprises:
a basic course educational material storage to store normal material about a chronic disease, the normal material being commonly provided to all patients;
a behavior correction educational material storage to store material about behaviors to be corrected, the material being provided to individual patients according to the information stored in the patient information storage; and
a complication prevention educational material storage to store material about complications, the material being provided to individual patients according to the information stored in the patient information storage.

16. The apparatus of claim 13, wherein the educational material selector comprises:
a complication management unit to calculate a risk to a complication of the patient based on at least one of the genetic information and the medical information and to select educational material appropriate for the patient; and
a lifestyle management unit to analyze the lifestyle of the patient based on the information about the health state and the lifestyle of the patient and to select educational material appropriate for the patient according to the analysis result.

17. The apparatus of claim 16, wherein the complication management unit calculates the risk to the complication of the patient based on at least one of the genetic information and the medical information, and selects complication prevention educational material when the risk to the complication is greater than or equal to a predetermined value.

18. The apparatus of claim 16, wherein the complication management unit controls a transmission frequency of the complication prevention educational material according to the risk to the complication.

19. The apparatus of claim 16, wherein the lifestyle management unit analyzes the lifestyle of the patient based on the information about the health state and the lifestyle of the patient and selects behavior correction educational material about the lifestyle according to the analysis result.

20. The apparatus of claim 19, wherein the lifestyle management unit controls a transmission frequency of the behavior correction educational material according to the analysis result.

21. The apparatus of claim 19, wherein the information about the health state of the patient comprises a blood sugar level of the patient, and the information about the lifestyle of the patient comprises at least one of meal menu and quantity, exercise type and time, and an attitude of the patient,
wherein the lifestyle management unit calculates at least one of a relationship between the meal menu and quantity and the blood sugar level of the patient and a relationship between the exercise type and time and the blood sugar level of the patient and prescribes meal menu and exercise appropriate for the patient according to the calculation result.
